(19) United States
(12) Patent Application Publication
(10) Pub. No.: US 2006/0099253 A1
(43) Pub. Date: May 11, 2006

(54) Antibiotic product formulation

(75) Inventors: Douglas C. Becker, Jamison, PA (US); Paul E. Stach, Plymouth, MN (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/252,640

(22) Filed: Oct. 18, 2005

Related U.S. Application Data
(60) Provisional application No. 60/620,565, filed on Oct. 20, 2004.

(51) Int. Cl. A61K 9/20; A61K 38/14; A61K 31/63; A61K 31/43; A61K 31/545; A61K 31/496

(52) U.S. Cl. 424/464; 514/192; 514/200; 514/8; 514/253.08; 514/155

(57) ABSTRACT

This invention discloses an antibiotic formulation in an immediate release antibiotic dosage form, comprising an antibiotic, colloidal silicon dioxide, povidone, silicified microcrystalline cellulose, croscarmellose sodium, magnesium stearate and a coating, said formulation having a release profile wherein the Cₘₐₓ is reached in less than five hours.

This application claims priority from copending provisional application Ser. No. 60/620,565, filed Oct. 20, 2004, the entire disclosure of which is hereby incorporated by reference.

### BACKGROUND OF THE INVENTION

This invention relates to an antibiotic product formulation.

A wide variety of antibiotics have been used, and will be used, in order to combat bacterial infection. In general, such antibiotics can be administered by a repeated dosing of immediate release dosage forms. The composition of the dosage form can impact the bioavailability of the antibiotic.

This invention provides a formulation that consistently produces an antibiotic product that meets predetermined specifications and quality attributes.

### BRIEF SUMMARY OF THE INVENTION

In accordance with an aspect of this invention there is provided an antibiotic dosage formulation comprising an antibiotic, colloidal silicon dioxide, povidone, silicified microcrystalline cellulose, croscarmellose sodium, magnesium stearate and optionally a coating, the formulation having a release profile wherein the Cₘₐₓ is reached in less than five hours.

### BRIEF DESCRIPTION OF THE SEVERAL VIEWS OF THE DRAWING

FIG. 1 is a graph of the dissolution of several antibiotic product formulations containing ethionamide as the active ingredient. The x-axis is the time in days. The y-axis is the % dissolution. The dissolution profiles of the formulations do not meet the dissolution specification desired.

FIG. 2-FIG. 5 contain tables of dissolution profiles of the antibiotic product formulation of this invention containing ethionamide as the active ingredient.

FIG. 6 is a graph of the Cₘₐₓ for the antibiotic product formulation of FIG. 1 (reference) and FIG. 2 (test). The x-axis is the time in hours. The y-axis is the concentration in picograms/mL.

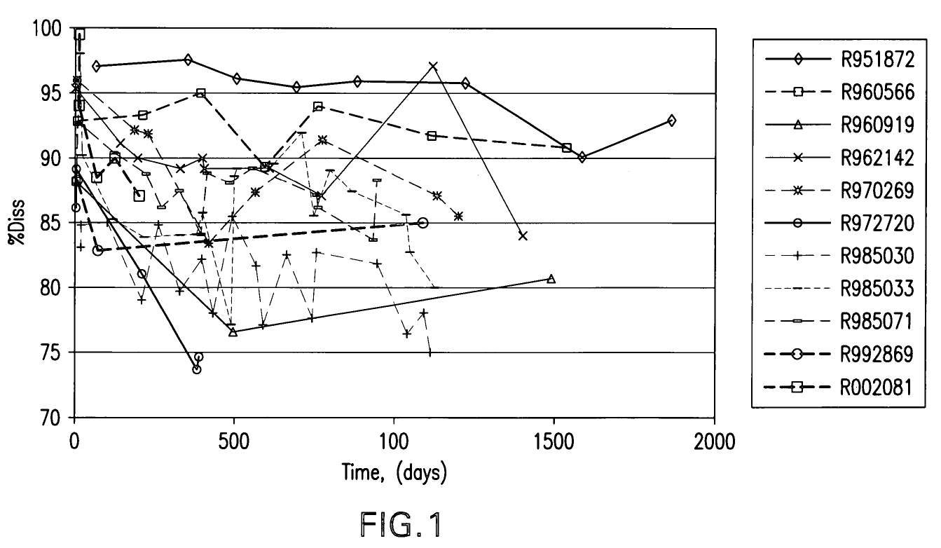

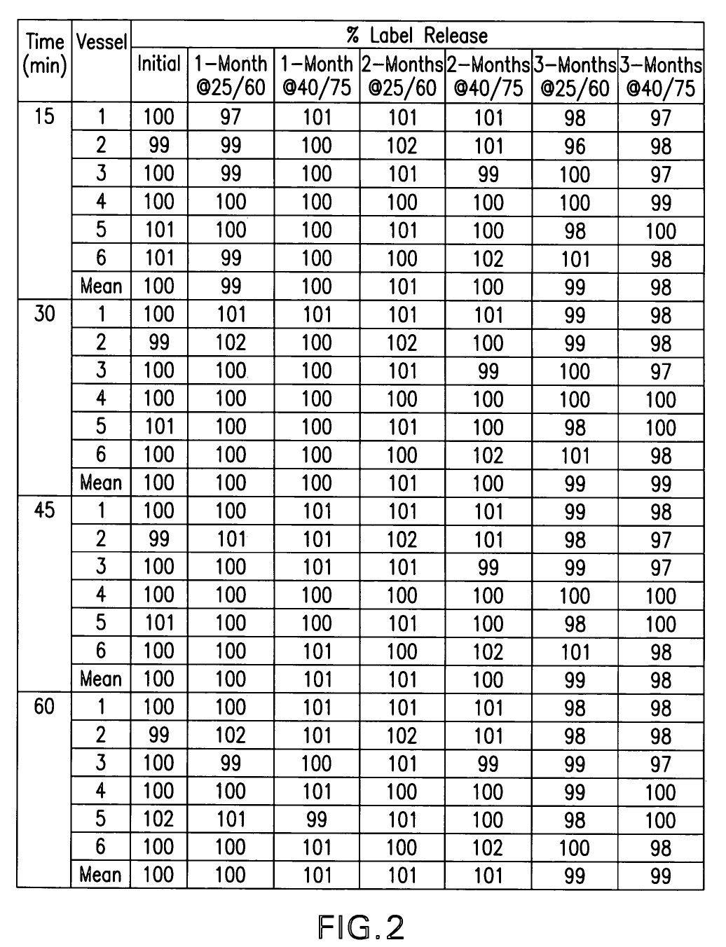

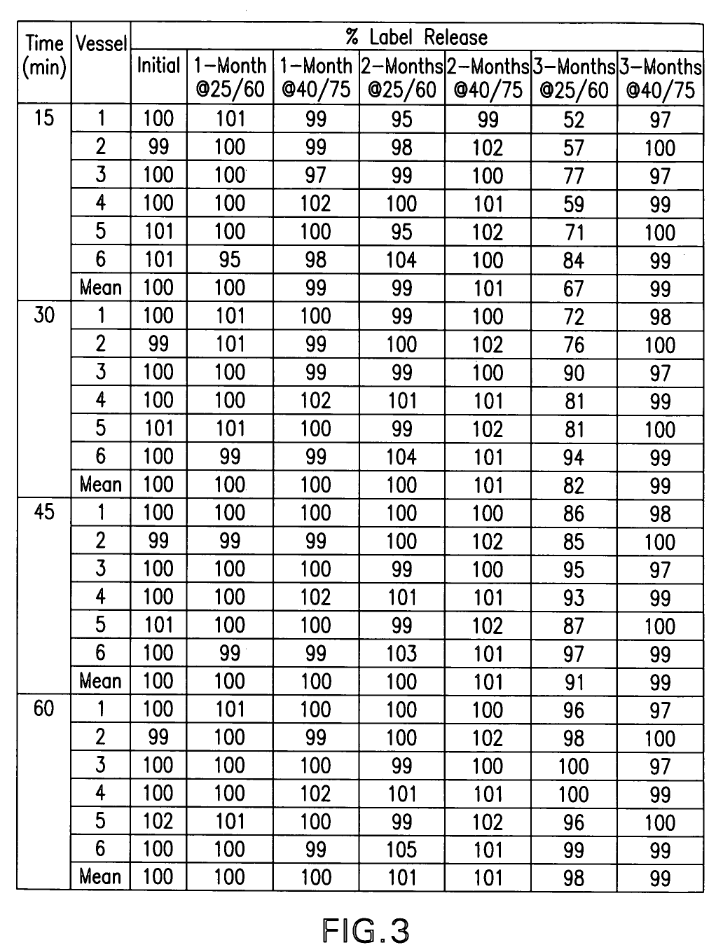

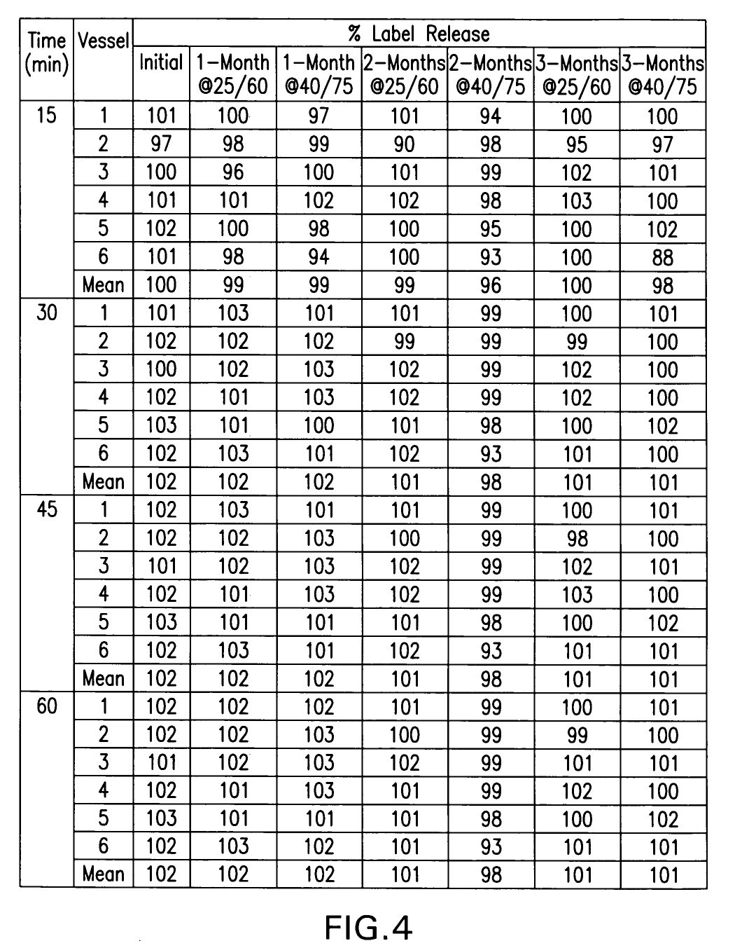

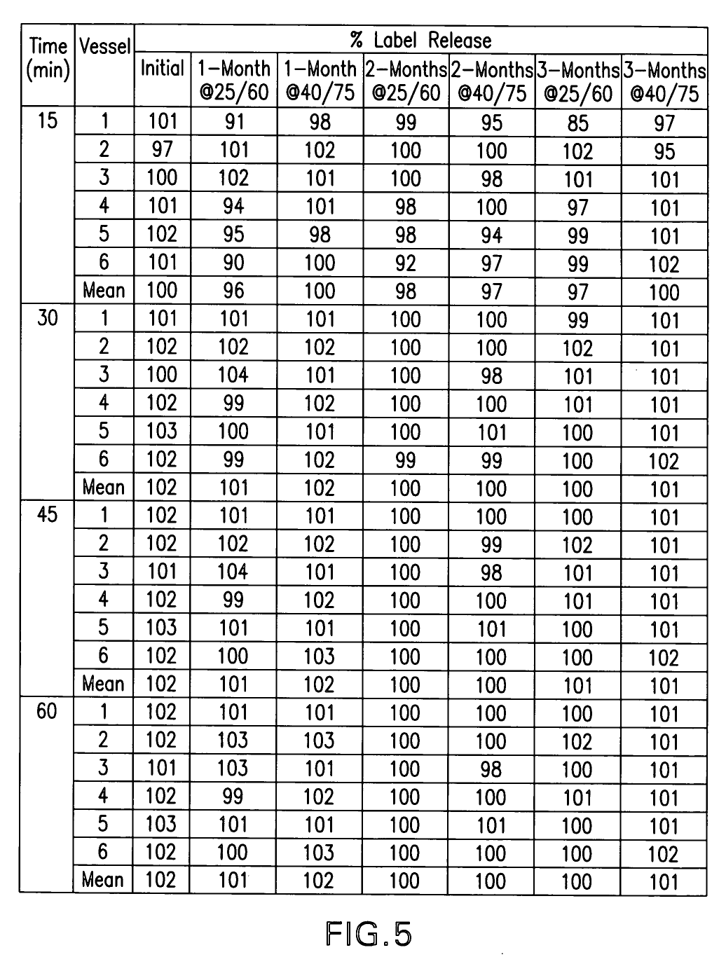

### DETAILED DESCRIPTION OF THE INVENTION

The following experimental details are set forth to aid in an understanding of the invention, and are not intended, and should not be construed, to limit in any way the invention set forth in the claims that follow thereafter.

In accordance with an aspect of this invention there is provided an antibiotic formulation in an immediate release antibiotic dosage form, comprising an antibiotic, colloidal silicon dioxide, povidone, silicified microcrystalline cellulose, croscarmellose sodium, magnesium stearate and a coating, the formulation having a release profile wherein the Cₘₐₓ is reached in less than five hours

The formulation will be especially useful in reaching a Cₘₐₓ in less than 5 hours. The following are examples of antibiotics that may be used in the formulation of this invention: cefadroxil, cefazolin, cephalexin, cephalothin, cephapirin, cephacelor, cephprozil, cephadrine, cefamandole, cefonicid, ceforanide, cefuroxime, cefixime, cefoperazone, cefotaxime, cefpodoxime, ceftaxidime, ceftibuten, ceftizoxime, ceftriaxone, cefepime, cefinetazole, cefotetan, cefoxitin, loracarbef, imipenem, erythromycin and salts thereof, azithromycin, clarithromycin, dirithromycin, troleanomycin, penicillin V penicillin salts and complexes, methicillin, nafcillin, oxacillin, cloxacillin, dicloxacillin, amoxicillin, amoxicillin and clavulanate potassium, ampicillin, bacampicillin, carbenicillin indanyl sodium, salts of carbenicillin, mezlocillin, piperacillin, tazobactam, ticarcillin, ticarcillin and clavulanate potassium, clindamycin, vancomycin, novobiocin, aminosalicyclic acid, capreomycin, cycloserine, ethambutol HCl and other salts, ethionamide, isoniazid, ciprofloxacin, levofloxacin, lomefloxacin, nalidixic acid, norfloxacin, ofloxacin, sparfloxacin, sulfacytine, sulfamerazine, sulfamethazine, sulfamethixole, sulfasalazine, sulfisoxazole, sulfapyrizine, sulfadiazine, sulfinethoxazole, sulfapyridine, metronidazole, methenamine, fosfomycin, nitrofurantoin, trimethoprim, clofazimine, co-triamoxazole, pentamidine, and trimetrexate.

An oral dosage form may comprise an antibiotic, e.g., ethionamide, in combination with one or more of a pharmaceutically acceptable lubricant, binder, and carrier. The oral dosage form is a tablet

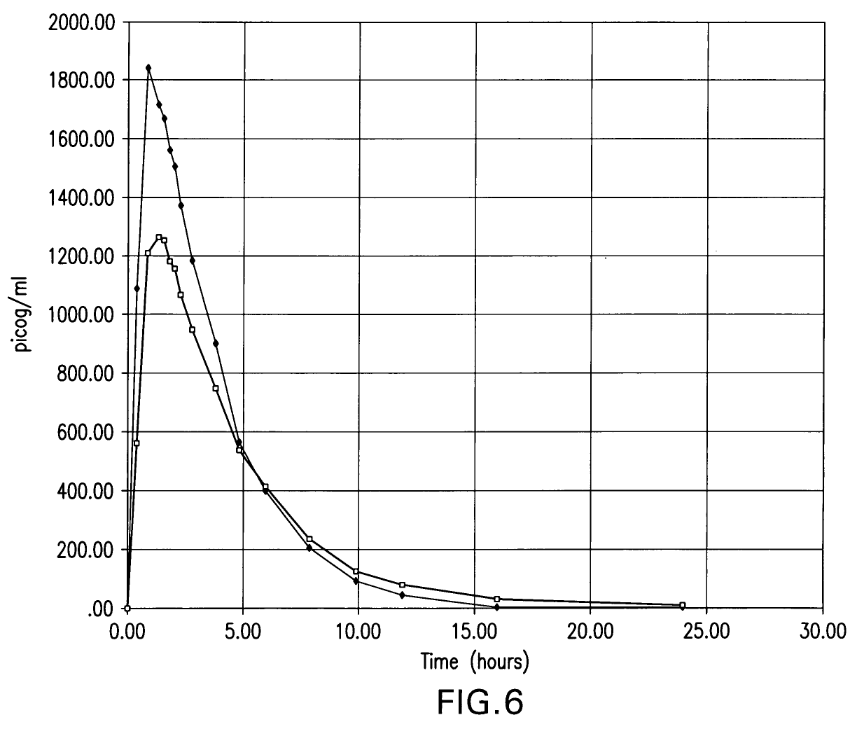

Cₘₐₓ was determined from the graph in FIG. 6 and is the highest concentration at any given dose appearing on the graph.

In an embodiment of this invention the antibiotic comprises up to about 42.0% w/w of the uncoated tablet. Other examples include from about 40 to about 45% (w/w) of antibiotic such as ethionamide.

In one embodiment colloidal silicon dioxide comprises up to about 0.5% w/w of the uncoated tablet. Examples include from 0.4 to about 0.6% (w/w) colloidal silicon dioxide.

In other embodiments povidone is up to about 5.0% w/w of the uncoated tablet. For example povidone comprises from about 3 to about 7% w/w of the uncoated tablet.

In further embodiments silicified microcrystalline cellulose comprises up to about 47.0% w/w of the uncoated tablet. For example silicified microcrystalline cellulose comprises from about 42 to about 50% w/w of the uncoated tablet.

Croscarmellose sodium may be included up to 5.0% w/w of the uncoated tablet. For example croscarmellose sodium comprises from about 3 to about 7% w/w of the uncoated tablet.

In yet further embodiments magnesium stearate comprises up to about 0.5% w/w of the uncoated tablet, e.g., from about 0.3 to about 0.7% w/w of the uncoated tablet.

The coating may for example be applied to give an increase in weight of about 4% (w/w) of the core or uncoated tablet.

The formulation is administered to a host in an amount effective for treating a bacterial infection. Any bacteria that is not considered normal flora in the host may cause the bacterial infection. An example of a bacterial infection includes, but is not limited to, tuberculosis.

The antibiotic formulation of the present invention may be initially produced and then coated to produce a form to give the desired Cₘₐₓ.

The coating is any coating that will produce a form to give the desired Cₘₐₓ. Opadry II Orange is an example of a coating that may be used.

### EXAMPLE FORMULATION

| | Amount per | % | Quantity per Batch |
|---|---|---|---|
| Ingredient | Tablet (mg) | (w/w) | (90,000) (kg) |
| Ethionamide USP | 250.0 | 42.0 | 22.5 |
| Colloidal Silicon Dioxide | 3.0 | 0.5 | 0.27 |
| Povidone USP K 29/32 | 30.0 | 5.0 | 2.70 |
| Silicified Microcrystalline | 284.0 | 47.0 | 25.56 |
| Cellulose NF | | | |
| Croscarmellose Sodium NF | 30.0 | 5.0 | 2.70 |
| Magnesium Stearate NF | 3.0 | 0.5 | 0.27 |
| Total Core Weight | 600.0 | 100 | 54.00 |
| Opadry II Orange 85F13774 | 24.0 | | 10.80* |
| Sterile Water for Irrigation, | removed | | 8.64 |
| USP | | | |
| Total | 624.0 | | |

*Represents a 20% solids dispersion in water

The ingredients found in the table above were screened in the following order to delump the ingredients: one half of the silicified microcrystalline cellulose, povidone, croscarmellose sodium, ethionamide, colloidal silicon dioxide, and the remainder of the silicified microcrystalline cellulose through a 20-mesh screen. The screened ingredients were transferred into a 5 cubic foot cross-flow V-blender and mixed for 20 minutes.

The magnesium stearate was passed through a NF through a 40-mesh screen and added through one blender charging port to the mixed powders. The powders were blended for 3 minutes. The blends were compressed into tablets. The tablets were acceptable if the target weight of 570-630 mg±5%, the dissolution of not less than 90% in 45 minutes, and the friability of less than 0.5%, were met.

Opadry Orange 85F13774 in sterile water for irrigation, USP, was dispensed and the above tablets were color coated using a Vector HCT-60 (24″/60 cm pan) at an inlet air temperature of 65° C.±10° C. and an outlet air temperature of 48° C.±5° C.

What is claimed is:

1. An antibiotic formulation comprising an antibiotic, colloidal silicon dioxide, povidone, silicified microcrystalline cellulose, croscarmellose sodium, magnesium stearate and a coating, said formulation having a release profile wherein the Cₘₐₓ is reached in less than five hours.

2. The formulation of claim 1 wherein the antibiotic is cefadroxil, cefazolin, cephalexin, cephalothin, cephapirin, cephacelor, cephprozil, cephadrine, cefamandole, cefonicid, ceforanide, cefuroxime, cefixime, cefoperazone, cefotaxime, cefpodoxime, ceftaxidime, ceftibuten, ceftizoxime, ceftriaxone, cefepime, cefinetazole, cefotetan, cefoxitin, loracarbef, imipenem, erythromycin and salts thereof, azithromycin, clarithromycin, dirithromycin, troleanomycin, penicillin V penicillin salts and complexes, methicillin, nafcillin, oxacillin, cloxacillin, dicloxacillin, amoxicillin, amoxicillin and clavulanate potassium, ampicillin, bacampicillin, carbenicillin indanyl sodium, salts of carbenicillin, mezlocillin, piperacillin, tazobactam, ticarcillin, ticarcillin and clavulanate potassium, clindamycin, vancomycin, novobiocin, aminosalicyclic acid, capreomycin, cycloserine, ethambutol HCl and other salts, ethionamide, isoniazid, ciprofloxacin, levofloxacin, lomefloxacin, nalidixic acid, norfloxacin, ofloxacin, sparfloxacin, sulfacytine, sulfamerazine, sulfamethazine, sulfamethixole, sulfasalazine, sulfisoxazole, sulfapyrizine, sulfadiazine, sulfinethoxazole, sulfapyridine, metronidazole, methenamine, fosfomycin, nitrofurantoin, trimethoprim, clofazimine, co-triamoxazole, pentamidine, and trimetrexate.

3. The formulation of claim 1 wherein the antibiotic is ethionamide.

4. The formulation of claim 1 wherein said formulation is in an oral dosage form.

5. The formulation of claim 4 wherein said formulation is a tablet.

6. The formulation of claim 5 wherein the tablet the coating is Opadry II Orange.

7. The formulation of claim 5 wherein the tablet has a dissolution of ≧90% in 45 minutes.

8. The formulation of claim 5 wherein the tablet has a friability of <0.5%.

9. The formulation of claim 5 wherein the uncoated tablet has a target weight of 570-630 mg±5%.

10. The formulation of claim 1 wherein the antibiotic is up to 42.0% w/w of the uncoated tablet.

11. The formulation of claim 1 wherein the colloidal silicon dioxide is up to 0.5% w/w of the uncoated tablet.

12. The formulation of claim 1 wherein the povidone is up to 5.0% w/w of the uncoated tablet.

13. The formulation of claim 1 wherein the silicified microcrystalline cellulose is up to 47.0% w/w of the uncoated tablet.

14. The formulation of claim 1 wherein the croscarmellose sodium is up to 5.0% w/w of the uncoated tablet.

15. The formulation of claim 1 wherein the magnesium stearate is up to 0.5% w/w of the uncoated tablet.

16. A method for treating a bacterial infection in a host comprising administering to the host the antibiotic formulation of claim 1.

17. The method of claim 16 wherein the bacterial infection is tuberculosis.
